(19) United States
(12) Patent Application Publication
(10) Pub. No.: US 2023/0338277 A1
(43) Pub. Date: Oct. 26, 2023

(54) CETRORELIX INJECTABLE LIQUID FORMULATION

(71) Applicant: BELOTECA, INC., San Diego, CA (US)

(72) Inventors: Frederik Defesche, San Diego, CA (US); Dorla Mirejovsky, San Diego, CA (US); Jenny Tran, San Diego, CA (US)

(73) Assignee: BELOTECA, INC., San Diego, CA (US)

(21) Appl. No.: 18/137,351

(22) Filed: Apr. 20, 2023

Related U.S. Application Data
(60) Provisional application No. 63/332,704, filed on Apr. 20, 2022.

(51) Int. Cl. A61K 9/00; A61K 9/08; A61K 38/09; A61K 47/02; A61K 47/12; A61K 47/26

(52) U.S. Cl. CPC A61K 9/0019; A61K 9/08; A61K 38/09; A61K 47/02; A61K 47/12; A61K 47/26

(57) ABSTRACT

Provided is an aqueous formulation comprising cetrorelix and/or a salt thereof, an isotonicity adjuster, a pH adjuster and water, suitable for subcutaneous injection. Also provided is a method of treating a patient with cetrorelix comprising injecting the above aqueous formulation to the patient in a manner sufficient to treat the patient.

### CROSS-REFERENCE TO RELATED APPLICATIONS

This application claims the benefit of U.S. Provisional Application No. 63/332,704, filed Apr. 20, 2022, and incorporated by reference herein in its entirety.

### BACKGROUND OF THE INVENTION

### (1) Field of the Invention

The present application generally relates to pharmaceutical formulations. More specifically, aqueous formulations of cetrorelix are provided that are stable for long periods of time.

### (2) Description of the Related Art

Cetrorelix acetate (below) is a synthetic decapeptide with gonadotropin-releasing hormone (GnRH) antagonistic activity. Cetrorelix acetate is an analog of native GnRH with substitutions of amino acids at positions 1, 2, 3, 6, and 10. The molecular formula is Acetyl-D-3-(2′-naphtyl)-alanine-D-4-chlorophenylalanine-D-3-(3′-pyridyl)-alanine-L-serine-L- tyrosine-D-citruline-L-leucine-L-arginine-L-proline-D-alanine-amide, and the molecular weight is 1431.06, calculated as the anhydrous free base. The structural formula is as follows:

### Cetrorelix Acetate

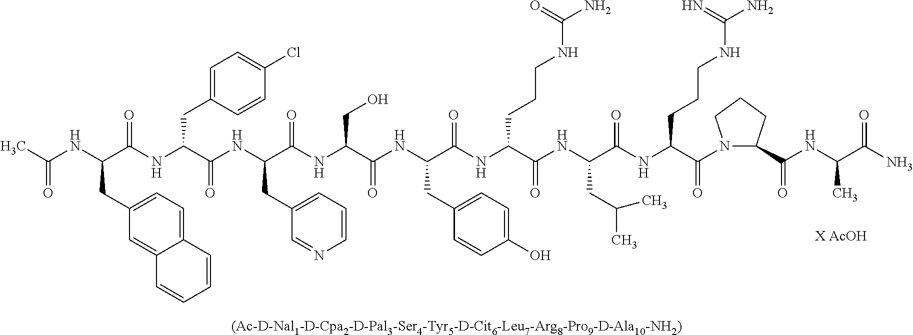

Cetrorelix acetate for injection is available as Cetrotide® by EMD Serono, Inc. for use in in-vitro fertilization, for which it is injected subcutaneously daily after follicle stimulation has been initiated and evidence of follicle maturation is approaching. Under those conditions, cetrorelix prevents an endogenous LH surge that would trigger an untimely ovulation prior to hCG administration.

Cetrotide® is supplied as a sterile lyophilized powder intended for subcutaneous injection after reconstitution with Sterile Water for Injection, USP, that is provided in a 1.0-mL pre-filled syringe. Each vial of Cetrotide® 0.25 mg contains 0.26-0.27 mg cetrorelix acetate, equivalent to 0.25 mg cetrorelix, and 54.80 mg mannitol. It is stored at 2-8° C., protected from light.

Cetrotide® is packaged in a tray containing one glass vial with lyophilized product, one pre-filled glass syringe with 1 mL of sterile water for injection, one 20-gauge needle for reconstitution of the lyophilized product with SWFI, and one 27-gauge needle for subcutaneous administration.

A cetrorelix acetate formulation filled directly in a syringe ready for administration would be far more beneficial to the end-user than the current Cetrotide® product due to its improved ease of use and less chance of error by the end-user. The present invention provides such a formulation.

### BRIEF SUMMARY OF THE INVENTION

Provided is an aqueous formulation comprising cetrorelix and/or a salt thereof, an isotonicity adjuster, a pH adjuster and water, suitable for subcutaneous injection. In these embodiments, the aqueous formulation comprises less than 0.5% deamidated cetrorelix (Ac-D-2-Nal-D-Cpa-D-Pal-Ser-Tyr-D-Cit-Leu-Arg-Pro-D-Ala-OH) after storage for at least one month at 25° C.

Also provided is a method of treating a patient with cetrorelix comprising injecting the above aqueous formulation to the patient in a manner sufficient to treat the patient.

### DETAILED DESCRIPTION OF THE INVENTION

Provided herewith is an aqueous formulation of cetrorelix that can be stored for months at room temperature without significant degradation into its deamidated degradant.

Thus, in some embodiments, an aqueous formulation comprising cetrorelix and/or a salt thereof, an isotonicity adjuster, a pH adjuster and water, that is suitable for subcutaneous injection is provided. In these embodiments, the aqueous formulation further comprises less than 0.5% deamidated cetrorelix (Ac-D-2-Nal-D-Cpa-D-Pal-Ser-Tyr-D-Cit-Leu-Arg-Pro-D-Ala-OH) after storage for at least one month at 25° C.

Exemplary salts of cetrorelix include but are not limited to the following: acetate, adipate, alginate, citrate, aspartate, benzoate, benzenesulfonate, bisulfate, butyrate, camphorate, camphorsulfonate, digluconate, cyclopentanepropionate, dodecylsulfate, ethanesulfonate, glucoheptanoate, glycerophosphate, hemisulfate, heptanoate, hexanoate, fumarate, hydrochloride, hydrobromide, hydroiodide, 2-hydroxy-ethanesulfonate, lactate, maleate, mesylate, methanesulfonate, nicotinate, 2-naphthalenesulfonate, oxalate, palmoate, pectinate, persulfate, 3-phenylpropionate, picrate, pivalate, propionate, succinate, tartrate, thiocyanate, tosylate, trifluoroacetate, and undecanoate. In some embodiments, the salt is cetrorelix acetate.

As established in the Example below, these formulations can be stored for at least four months at 25° C. without significant (0.5% or greater) degradation of the cetrorelix into its deamidated degradant. Thus, in some embodiments, the formulation comprises less than 0.5% of deamidated cetrorelix after storage for at least two months at 25° C. In some of those embodiments, the formulation comprises less than 0.5% of deamidated cetrorelix after storage for at least three months at 25° C. In other embodiments, the formulation comprises less than 0.5% of deamidated cetrorelix after storage for at least four months at 25° C.

The formulations of the present invention are not narrowly limited to any particular isotonicity adjuster. Suitable isotonicity adjusters include lactose, trehalose, dextrane sucrose, glycine, arginine, mannitol, sodium chloride, potassium chloride, glucose, polyethylene glycol, glycerin, sodium sulfate, sodium phosphate, potassium sodium tartrate, and sodium tartrate. In some embodiments, the isotonicity adjuster is mannitol.

The formulations of the present invention are also not narrowly limited to the use of any particular pH adjuster. In some embodiments, the pH adjuster is a base, e.g., aluminum hydroxide, calcium hydroxide, manganese hydroxide, potassium hydroxide, ammonium hydroxide, magnesium hydroxide, sodium hydroxide, or zinc hydroxide. See, e.g., www.endmemo.com/chem/baseslist.php. In other embodiments, the pH adjuster is an acid. Nonlimiting examples of suitable acids are malonic acid, citric acid, tartartic acid, glutamic acid, phthalic acid, azelaic acid, barbituric acid, benzilic acid, cinnamic acid, fumaric acid, glutaric acid, gluconic acid, hexanoic acid, lactic acid, malic acid, oleic acid, folic acid, propiolic acid, propionic acid, rosolic acid, stearic acid, tannic acid, trifluoroacetic acid. uric acid, ascorbic acid, gallic acid, acetylsalicylic acid, acetic acid, sulfurous acid, sulfuric acid, hyposulfurous acid, persulfuric acid, pyrosulfuric acid, disulfurous acid, dithionous acid, tetrathionic acid, thiosulfurous acid, hydrosulfuric acid, peroxydisulfuric acid, perchloric acid, hydrochloric acid, hypochlorous acid, chlorous acid, chloric acid, hyponitrous acid, nitrous acid, nitric acid, pernitric acid, carbonous acid, carbonic acid, hypocarbonous acid, percarbonic acid, oxalic acid, acetic acid, phosphoric acid, phosphorous acid, hypophosphous acid, perphosphoric acid, hypophosphoric acid, pyrophosphoric acid, hydrophosphoric acid, hydrobromic acid, bromous acid, bromic acid, hypobromous acid, hypoiodous acid, iodous acid, iodic acid, periodic acid, hydroiodic acid, fluorous acid, fluoric acid, hypofluorous acid, perfluoric acid, hydrofluoric acid, chromic acid, chromous acid, hypochromous acid, perchromic acid, hydroselenic acid, selenic acid, selenous acid, hydronitric acid, boric acid, molybdic acid, perxenic acid, silicofluoric acid, telluric acid, tellurous acid, tungstic acid, xenic acid, formic acid, pyroantimonic acid, permanganic acid, manganic acid, antimonic acid, antimonous acid, silicic acid, titanic acid, arsenic acid, pertechnetic acid, hydroarsenic acid, dichromic acid, tetraboric acid, metastannic acid, hypooxalous acid, ferricyanic acid, cyanic acid, silicous acid, hydrocyanic acid, thiocyanic acid, uranic acid, and diuranic acid, as those compounds are commonly known. See, e.g., www.endmemo.com/chem/acidslist.php. The suitability of any particular acid for adjusting the pH of a particular cetrorelix formulation can be determined without undue experimentation. In specific embodiments, pH adjuster is acetic acid.

The aqueous formulations of these embodiments can have any pH. In some embodiments, the aqueous formulation has a pH of 2.5-4.0.

The invention aqueous formulations can be packaged in any form. In some embodiments, they are packaged in a syringe, with or without a needle. In other embodiments, they are packaged in a vial.

The cetrorelix in the invention aqueous formulations can be at any concentration. In some embodiments, the cetrorelix concentration is 0.20-0.30 mg/mL.

The present invention is also directed to a method of treating a patient with cetrorelix. The method comprises injecting any of the aqueous formulations discussed above to the patient in a manner sufficient to treat the patient. The formulation can be injected in any manner. In some embodiments, the injection is subcutaneous, e.g., into the abdomen.

Preferred embodiments are described in the following examples. Other embodiments within the scope of the claims herein will be apparent to one skilled in the art from consideration of the specification or practice of the invention as disclosed herein. It is intended that the specification, together with the examples, be considered exemplary only, with the scope and spirit of the invention being indicated by the claims, which follow the examples.

### EXAMPLE 1

### Stability Studies with Cetrorelix Acetate

The stability of cetrorelix formulations were evaluated. Initially, a bulk solution as shown in Table 1 was filled in 2 mL/13 mm glass Type I vials and lyophilized. The lyophilized product was placed storage conditions of 25° C. and at the recommended storage of 2-8° C. The lyophilized product contained large number of impurities matching those found in Cetrotide®.

TABLE 1
Composition of Bulk Solution

| Ingredient | Function | Concentration |
|---|---|---|
| Cetrorelix¹ | Active Ingredient | 0.25 mg/mL |
| Mannitol, USP | Bulking Agent | 54.80 mg/mL |
| Purified Water | Diluent | Q.S. to 1 mL |

¹As Cetrorelix free and anhydrous base

Testing of several lots Cetrotide® showed one main degradant, deamidated alanine at position Ci₁₀ leading to a free acid (Table 2).

TABLE 2
Testing of Various Lots of Cetrotide ® at the Expiry

| | Cetrotide | | |
|---|---|---|---|
| | Lot P00339A | Lot 9H042D | Lot P00494D |
| HPLC Analysis | Exp. October 2019 | Exp. July 2021 | Exp. July 2021 |
| Assay, % LC | 105 | 100.5 | 99.9 |

Cetrorelix Related Substances, % w/w

| | | | | |
|---|---|---|---|---|
| 1. | Specified | 0.64 | 0.21 | 0.41 |
| | Identified | | | |
| | Degradation | | | |
| | Product | | | |
| a. | Deamidation | | | |
| | Impurity ¹ | | | |
| 2. | Any Other | RRT 0.98-0.06 | RRT 0.97-0.13 | RRT 0.97-0.12 |
| | Unspecified | RRT 1.07-0.06 | RRT 1.04-0.05 | RRT 1.04-0.06 |
| | Degradation | RRT 1.34-0.05 | RRT 1.16-0.07 | RRT 1.16-0.06 |
| | Product | RRT 1.55-0.15 | | |
| 3. | Total | 0.96 | 0.46 | 0.65 |
| | Impurities | | | |

| | | |
|---|---|---|
| Date of Testing | 28 Oct. 2019 | 6 Jul. 2021 |

¹ Ac-D-2-Nal-D-Cpa-D-Pal-Ser-Tyr-D-Cit-Leu-Arg-Pro-D-Ala-OH

By contrast, the bulk solution stored at 25° C. and 40° C. unexpectedly remained stable, as shown in Table 3.

TABLE 3
Growth of Deamidation Degradant at 25°
C. and 40° C. over 8 Days

| | Number | % Impurity (RRT 1.1) | |
|---|---|---|---|
| Temperature | of Days | 0.1% Acetic Acid | 0.25% Acetic Acid |
| 25° C. | 0 | ND | ND |
| | 2 | ND | ND |
| | 8 | <LOQ | 0.07 |
| 40° C. | 0 | ND | ND |
| | 2 | 0.08 | 0.14 |
| | 8 | 0.14 | 0.37 |

This unexpected discovery led us to postulate that the development of a liquid product could be feasible.

Based on the initial discovery, an additional bulk solution was prepared according to the composition shown in Table 4, filtered, and filled as a 1-mL fill into 2-mL glass vials and into 1-mL syringes. The container closures selected for the study are listed in Table 5.

TABLE 4
Bulk Solution Composition for Cetrorelix Injection, 0.25 mg/mL

| Ingredient | Function | Concentration | |
|---|---|---|---|
| Cetrorelix¹ | Active Ingredient | 0.25 | mg/mL |
| Mannitol, USP | Bulking Agent | 54.80 | mg/mL |
| Glacial Acetic Acid | pH Adjuster | 1 | mg/mL |

| | | |
|---|---|---|
| Water for Injection, USP | Diluent | Q.S. to 1 mL |

¹As Cetrorelix acetate (0.26-0.27 mg/mL)

TABLE 5
Primary Packaging Components for Cetrorelix Injection

| Component | Supplier |
|---|---|
| 2R Type 1 Clear Glass Vial | OMPI |
| 13 mm Stopper Bromobutyl Coated - | West Pharmaceutical Services |
| 1358 4023/50G | |
| Syringe EZ-Fill 1 mL Long ITC 7025/65 | OMPI |
| Plunger 2340 4023/50 Gray B2-40 | West Pharmaceutical Services |

The filled containers were placed at 2-8° C. and 25° C. Vials were placed in an upright and inverted orientations, syringes in a horizontal position.

Stability data obtained up to 4 months are presented in Table 6 (vials, 25° C.), Table 7 (vials 2-8° C.) and Table 8 (syringes). The data show that cetrorelix injection stored at 2-8° C. for 4 months does not contain any detectable degradant. At 25° C., two degradants were obtained, the expected deamidation cetrorelix and a degradant with a retention time similar to a degradant obtained during the force degradation studies by heat.

TABLE 6
Stability of Cetrorelix Injection in 2R Vials Stored at 25° C. for 4 Months

| | Time Point | | | | | | | |
|---|---|---|---|---|---|---|---|---|
| | 1 Month | | 2 Months | | 3 Months | | 4 Months | |
| | 6 Jan. 2022 | | 7 Feb. 2022 | | 7 Mar. 2022 | | 7 Apr. 2022 | |
| Test | Upright | Inverted | Upright | Inverted | Upright | Inverted | Upright | Inverted |
| Physical Appearance | Clear, | Clear, | Clear, | Clear, | Clear, | Clear, | Clear, | Clear, |
| | colorless | colorless | colorless | colorless | colorless | colorless | colorless | colorless |
| | solution free | solution free | solution free | solution free | solution free | solution free | solution free | solution free |
| | of visible | of visible | of visible | of visible | of visible | of visible | of visible | of visible |
| | particulates | particulates | particulates | particulates | particulates | particulates | particulates | particulates |
| Assay, % Label Claim | 98.4 | 98.2 | 96.4 | 96.4 | 97.3 | 97.0 | 97.7 | 97.4 |
| (0.25 mg/mL) | | | | | | | | |

Related Substances via HPLC (w/w %)

| | | | | | | | | |
|---|---|---|---|---|---|---|---|---|
| Specified-Identified | 0.08 | 0.15 | 0.16 | 0.23 | 0.27 | 0.26 | 0.40 | 0.35 |
| Degradation Product | | | | | | | | |
| a) Deamidation Impurity | | | | | | | | |
| Any Unidentified | RRT 0.86- | RRT 0.86- | RRT 0.86- | RRT 0.86- | RRT 0.86- | RRT 0.86- | RRT 0.86- | RRT 0.86- |
| Unspecified | 0.08 | 0.10 | 0.16 | 0.17 | 0.22 | 0.23 | 0.29 | 0.30 |
| Degradation Product | | | | | | | | |
| Total Related Substances | 0.19 | 0.25 | 0.38 | 0.41 | 0.49 | 0.49 | 0.69 | 0.65 |
| pH | 3.34 | 3.33 | NT | NT | 3.40 | 3.40 | NT | NT |

TABLE 7
Stability of Cetrorelix Injection in 2R Vials Stored at 2-8° C. for 4 Months

| | Time Point | | | | | |
|---|---|---|---|---|---|---|
| | 1 Month | | 3 Months | | 4 Months | |
| | 6 Jan. 2022 | | 7 Mar. 2022 | | 7 Apr. 2022 | |
| Test | Upright | Inverted | Upright | Inverted | Upright | Inverted |
| Physical Appearance | Clear, | Clear, | Clear, | Clear, | Clear, | Clear, |
| | colorless | colorless | colorless | colorless | colorless | colorless |
| | solution free | solution free | solution free | solution free | solution free | solution free |
| | of visible | of visible | of visible | of visible | of visible | of visible |
| | particulates | particulates | particulates | particulates | particulates | particulates |
| Assay, % Label Claim | 99.6 | 101.3 | 98.0 | 97.6 | 98.9 | 98.0 |
| (0.25 mg/mL) | | | | | | |

Related Substances via HPLC (w/w %)

| | | | | | | |
|---|---|---|---|---|---|---|
| Specified-Identified | ND | ND | ND | ND | ND | ND |
| Degradation Product | | | | | | |
| a) Deamidation | | | | | | |
| Any Unidentified | ND | ND | ND | ND | ND | ND |
| Unspecified | | | | | | |
| Degradation Product | | | | | | |
| Total Related Substances | ND | ND | ND | ND | ND | ND |
| pH | 3.31 | 3.28 | 3.26 | 3.26 | NT | NT |

ND = Not Detected
NT = Not Tested

TABLE 8
Stability of Cetrorelix Injection Filled in Syringes and Stored at 2-8° C. and 25° C. for 4 Months

| | Time Point | | | | | | |
|---|---|---|---|---|---|---|---|
| | 1 Month | | 2 Months | 3 Months | | 4 Months | |
| | 6 Jan. 2022 | | 8 Feb. 2022 | 7 Mar. 2022 | | 7 Apr. 2022 | |
| Test | 2-8° C. | 25° C. | 25° C. | 2-8° C. | 25° C. | 2-8° C. | 25° C. |
| Physical Appearance | Clear, colorless | Clear, colorless | Clear, colorless | Clear, colorless | Clear, colorless | Clear, colorless | Clear, colorless |
| | solution free of | solution free of | solution free of | solution free of | solution free of | solution free of | solution free of |
| | visible | visible | visible | visible | visible | visible | visible |
| | particulates | particulates | particulates | particulates | particulates | particulates | particulates |
| Assay, % Label Claim | 100.7 | 100.0 | 96.8 | 95.6 | 97.2 | 98.0 | 97.5 |
| (0.25 mg/mL) | | | | | | | |

Related Substances via HPLC (w/w %)

| | | | | | | | |
|---|---|---|---|---|---|---|---|
| Specified-Identified | ND | 0.12 | 0.18 | ND | 0.35 | ND | 0.48 |
| Degradation Product | | | | | | | |
| a) Deamidation | | | | | | | |
| Any Unidentified | ND | RRT 0.86-0.10 | RRT 0.86-0.18 | ND | RRT 0.86-0.24 | ND | RRT 0.62-0.07 |
| Unspecified | | | | | | | RRT 0.86-0.30 |
| Degradation Product | | | | | | | |
| Total Related Substances | ND | 0.22 | 0.37 | ND | 0.59 | ND | 0.85 |
| pH | 3.38 | 3.34 | NT | 3.27 | 3.44 | NT | NT |

ND = Not Detected
NT—Not Tested

### EXAMPLE 2

### Long Term Stability Studies with Cetrorelix Acetate

The stability presented for a composition and container closure systems shown in Table 4 and Table 5 was continued over 6 months of storage at 25° C. as presented in Table 6 (2R vials upright and inverted orientations) and Table 10 (syringes). The stability results for vials stored at the recommended storage conditions of 2-8° C. over 15.5 months are shown in Table 11 (upright orientation) and Table 12 (inverted orientation). The stability data over 15.5 months for syringes are summarized Table 13.

The data show that cetrorelix injection stored at 2-8° C. for 15.5 months in vials or syringes contains the expected deamidation cetrorelix at a level of 0.11% - 0.13%. This degradant was observed for the first time at 15.5 months. It is expected that at the proposed expiration time of 24 months the level of the deamidated cetrorelix will be below 0.5%. At 25° C., two main degradants were obtained, the expected deamidation cetrorelix and a degradant with a retention time similar to a degradant obtained during the force degradation studies by heat. After 6 months of storage at 25° C. both degradants are reaching the levels close to 0.5%. The level of the deamidated cetrorelix was higher at 6 months in a syringe reaching a level of 0.76%. As cetrorelix is a very heat sensitive compound, formation of higher level of an impurity under accelerated conditions is not uncommon.

To elaborate further on the effect of acetic acid content on cetrorelix stability, a study was designed to test the stability of cetrorelix as a function of acetic acid content: 0.25% w/v, 0.5% w/v, and 0.8%. As described in US Patent Application Publication 20020099018, to facilitate the filtration of cetrorelix, cetrorelix bulk solution must be acidified. See also Powell, M. F., Pharmaceutical Research 8:1258 (1991); Dathe, M: Int. J. Peptide Protein Res. 36:344-349 (1990); Szejtli, J.: Pharmaceutical Technology International 16-22 (1991), establishing that oligopeptides, particularly those with terminal acid amide function, tend to form gels. During sterile filtration this is apparent from the speed of filtration; indeed the increased viscosity of such solutions can often already be detected organoleptically. A gelatinous layer remains on the sterile filter. It is then no longer possible to prepare a medication with an exactly and reproducibly defined active substance content.

Cetrorelix bulk solutions were prepared using a composition as shown in Table 4 with a varying content of glacial acid 2.5 mg/mL, 5 mg/mL, and 8 mg/mL, respectively. The container closures selected for the study are listed below:

| Component | Supplier |
|---|---|
| EZ-Fill 1 mL Long Syringe, 27G1/2″ | OMPI |
| needle | |
| Plunger W4023 Flur Daikyo SI1000 | West Pharmaceutical Services |

Syringes were placed on stability in a horizontal position at 25° C. and 2-8° C. Table shows the results for Cetrorelix formulated with 0.25% acetic acid and stored at 25° C. for 9 months, while the stability of Cetrorelix with 0.5% and 0.8% acetic acid are shown in Table. The testing of these two formulations ended after 3 months due to high formation of deamidated cetrorelix. The stability results for cetrorelix formulated with 0.25% acetic acid and stored at 2-8° C. for 11 months are shown in Table 16, while Table 17 summarizes the 2-8° C. data for cetrorelix injection with 0.5% and 0.8% acetic acid.

The study revealed that the formation of deamidated cetrorelix is enhanced by higher content of acetic acid. However, at the recommended storage temperature of 2-8° C., cetrorelix was stable in the presence of 0.25% and 0.5% acetic acid. In the presence of 0.25% acetic acid 0.16% of deamidated cetrorelix was formed at 11 months while none was yet detected at 6 months in the presence of 0.5% acetic acid.

Addition of acetic acid to cetrorelix injection lowers the pH. Therefore, another study was initiated to determine the stability of cetrorelix injection at higher pH. Cetrorelix injection was first compounded with 2.5 mg acetic acid, then the pH was adjusted to pH 4 with sodium hydroxide. The bulk solution after filtration was filled into the container closure systems shown above. Syringes were stored at 25° C. and 2-8° C. and tested as presented in Table (9 months at 25° C.) and Table 9 (11 months at 2-8° C.). No reportable related substances were found in samples stored at 2-8° C. for 11 months.

Surprising stability of cetrorelix at pH 4 led to additional examination of stability of cetrorelix at pH 4.5, pH 5.0, and pH 6.0. New batches were made with cetrorelix compounded with 1 mg/mL acetic acid followed by a pH adjustment with sodium hydroxide to pH 4.5, pH 5.0, and pH 6.0. Stability data for samples exposed to 25° C. for 4 months are summarized in Table 20. Table 21-23 present the stability of cetrorelix injection with adjusted pH to pH 4.5, pH 5.0, and pH 6.0 after a storage at the recommended storage temperature of 2-8° C. for 7.5 months, respectively.

The study indicates that at higher pH the deamidation degradation is much slower than at the acidic pH. At 25° C. the main degradant appears to be the heat degradant with RRT 0.86 seen also under other condition at 25° C. No reportable related substances were found in samples stored at 2-8° C. for 7.5 months.

TABLE 9
Stability of Cetrorelix Injection in 2R Vials Stored at 25° C. for 6 Months in Upright and Inverted Positions

| | Time Point | | | | | | | | | |
|---|---|---|---|---|---|---|---|---|---|---|
| | 1 Month | | 2 Months | | 3 Months | | 4 Months | | 6 Months | |
| | 7 Jan. 2022 | | 7 Feb. 2022 | | 7 Mar. 2022 | | 7 Apr. 2022 | | 7 Jun. 2022 | |
| Test | Upright | Inverted | Upright | Inverted | Upright | Inverted | Upright | Inverted | Upright | Inverted |
| Physical Appearance | Clear, | Clear, | Clear, | Clear, | Clear, | Clear, | Clear, | Clear, | Clear, | Clear, |
| | colorless | colorless | colorless | colorless | colorless | colorless | colorless | colorless | colorless | colorless |
| | solution | solution | solution | solution | solution | solution | solution | solution | solution | solution |
| | free of | free of | free of | free of | free of | free of | free of | free of | free of | free of |
| | visible | visible | visible | visible | visible | visible | visible | visible | visible | visible |
| | partic- | partic- | partic- | partic- | partic- | partic- | partic- | partic- | partic- | partic- |
| | ulates | ulates | ulates | ulates | ulates | ulates | ulates | ulates | ulates | ulates |
| Assay, % Label Claim | 98.4 | 98.2 | 96.4 | 96.4 | 97.3 | 97.0 | 97.7 | 97.4 | 98.0 | 99.4 |
| (0.25 mg/mL) | | | | | | | | | | |

Related Substances via HPLC (w/w %)

| | | | | | | | | | | |
|---|---|---|---|---|---|---|---|---|---|---|
| Specified-Identified | 0.08 | 0.15 | 0.16 | 0.23 | 0.27 | 0.26 | 0.40 | 0.35 | 0.50 | 0.51 |
| Degradation Product | | | | | | | | | | |
| b) Deamidation | | | | | | | | | | |
| Impurity | | | | | | | | | | |
| Any Unidentified | RRT 0.86- | RRT 0.86- | RRT 0.86- | RRT 0.86- | RRT 0.86- | RRT 0.86- | RRT 0.86- | RRT 0.86- | RRT 0.52- | RRT 0.52- |
| Unspecified | 0.08 | 0.10 | 0.16 | 0.17 | 0.22 | 0.23 | 0.29 | 0.30 | 0.07 | 0.09 |
| Degradation Product | | | | | | | | | RRT 0.86- | RRT 0.86- |
| | | | | | | | | | 0.48 | 0.49 |
| Total Related | 0.19 | 0.25 | 0.38 | 0.41 | 0.49 | 0.49 | 0.69 | 0.65 | 1.05 | 1.09 |
| Substances | | | | | | | | | | |
| pH | 3.34 | 3.33 | NT | NT | 3.40 | 3.40 | NT | NT | 3.45 | 3.44 |

TABLE 10
Stability of Cetrorelix Injection Filled in Syringes and Stored at 25° C. for 6 Months

| | Time Point | | | | |
|---|---|---|---|---|---|
| Test | 1 Month | 2 Months | 3 Months | 4 Months | 6 Months |
| | 7 Jan. 2022 | 7 Mar. 2022 | 7 Apr. 2022 | 7 Apr. 2022 | 7 Jun. 2022 |
| Physical Appearance | Clear, colorless | Clear, colorless | Clear, colorless | Clear, colorless | Clear, colorless |
| | solution free of | solution free of | solution free of | solution free of | solution free of |
| | visible particulates | visible particulates | visible particulates | visible particulates | visible particulates |
| Assay, % Label Claim | 100.0 | 96.8 | 97.2 | 97.5 | 99.8 |
| (0.25 mg/mL) | | | | | |

Related Substances via HPLC (w/w %)

| | | | | | |
|---|---|---|---|---|---|
| Specified-Identified | 0.12 | 0.18 | 0.35 | 0.48 | 0.76 |
| Degradation Product | | | | | |
| a) Deamidation | | | | | |
| Any Unidentified | RRT 0.86-0.10 | RRT 0.86-0.18 | RRT 0.86-0.24 | RRT 0.62-0.07 | RRT 0.52-0.07 |
| Unspecified | | | | RRT 0.86-0.30 | RRT 0.86-0.45 |
| Degradation Product | | | | | |
| Total Related Substances | 0.22 | 0.37 | 0.59 | 0.85 | 1.28 |
| pH | 3.34 | NT | 3.44 | NT | 3.42 |

NT = Not Tested

TABLE 11
Stability of Cetrorelix Injection in 2R Vials Stored Upright at 2-8° C. for 15.5 Months

| | Time Point | | | | | | |
|---|---|---|---|---|---|---|---|
| | 1 Month | 3 Months | 4 Months | 6 Months | 9 Months | 12 Months | 15.5 Months |
| Test | 7 Jan. 2022 | 7 Mar. 2022 | 7 Apr. 2022 | 7 Jun. 2022 | 7 Sep. 2022 | 7 Dec. 2022 | 21 Mar. 2023 |
| Physical Appearance | Clear, colorless | Clear, colorless | Clear, colorless | Clear, colorless | Clear, colorless | Clear, colorless | Clear, colorless |
| | solution free of | solution free of | solution free of | solution free of | solution free of | solution free of | solution free of |
| | visible | visible | visible | visible | visible | visible | visible |
| | particulates | particulates | particulates | particulates | particulates | particulates | particulates |
| Assay, % Label Claim | 99.6 | 98.0 | 98.9 | 101.0 | 95.4 | 96.4 | 95.1 |
| (0.25 mg/mL) | | | | | | | |

Related Substances via HPLC (w/w %)

| | | | | | | | |
|---|---|---|---|---|---|---|---|
| Specified-Identified | ND | ND | ND | ND | ND | ND | 0.10 |
| Degradation Product | | | | | | | |
| b) Deamidation | | | | | | | |
| Any Unidentified | ND | ND | ND | ND | ND | ND | ND |
| Unspecified | | | | | | | |
| Degradation Product | | | | | | | |
| Total Related Substances | ND | ND | ND | ND | ND | ND | 0.10 |
| pH | 3.31 | 3.26 | NT | 3.39 | 3.36 | NT | NT |

ND = Not Detected
NT = Not Tested

TABLE 12
Stability of Cetrorelix Injection in 2R Vials Stored Inverted at 2-8° C. for 15.5 Months

| | Time Point | | | | | | |
|---|---|---|---|---|---|---|---|
| | 1 Month | 3 Months | 4 Months | 6 Months | 9 Months | 12 Months | 15.5 Months |
| Test | 7 Jan. 2022 | 7 Mar. 2022 | 7 Apr. 2022 | 7 Jun. 2022 | 7 Sep. 2022 | 7 Dec. 2022 | 21 Mar. 2023 |
| Physical Appearance | Clear, colorless | Clear, colorless | Clear, colorless | Clear, colorless | Clear, colorless | Clear, colorless | Clear, colorless |
| | solution free of | solution free of | solution free of | solution free of | solution free of | solution free of | solution free of |
| | visible | visible | visible | visible | visible | visible | visible |
| | particulates | particulates | particulates | particulates | particulates | particulates | particulates |
| Assay, % Label Claim | 101.3 | 97.6 | 98.0 | 100.5 | 95.0 | 96.1 | 95.1 |
| (0.25 mg/mL) | | | | | | | |

Related Substances via HPLC (w/w %)

| | | | | | | | |
|---|---|---|---|---|---|---|---|
| Specified-Identified | ND | ND | ND | ND | ND | ND | 0.11 |
| Degradation Product | | | | | | | |
| a) Deamidation | | | | | | | |
| Any Unidentified | ND | ND | ND | ND | ND | ND | ND |
| Unspecified | | | | | | | |
| Degradation Product | | | | | | | |
| Total Related Substances | ND | ND | ND | ND | ND | ND | 0.11 |
| pH | 3.28 | 3.26 | NT | 3.40 | 3.37 | NT | NT |

ND = Not Detected
NT = Not Tested

TABLE 13
Stability of Cetrorelix Injection Filled in Syringes and Stored at 2-8° C. for 15.5 Months

| | Time Point | | | | | | |
|---|---|---|---|---|---|---|---|
| | 1 Month | 3 Months | 4 Months | 6 Months | 9 Months | 12 Months | 15.5 Months |
| Test | 7 Jan. 2022 | 7 Mar. 2022 | 7 Apr. 2022 | 7 Jun. 2022 | 7 Sep. 2022 | 7 Dec. 2022 | 21 Mar. 2023 |
| Physical Appearance | Clear, colorless | Clear, colorless | Clear, colorless | Clear, colorless | Clear, colorless | Clear, colorless | Clear, colorless |
| | solution free of | solution free of | solution free of | solution free of | solution free of | solution free of | solution free of |
| | visible | visible | visible | visible | visible | visible | visible |
| | particulates | particulates | particulates | particulates | particulates | particulates | particulates |
| Assay, % Label Claim | 100.7 | 95.6 | 98.0 | 101.0 | 95.7 | 96.9 | 95.4 |
| (0.25 mg/mL) | | | | | | | |

Related Substances via HPLC (w/w %)

| | | | | | | | |
|---|---|---|---|---|---|---|---|
| Specified-Identified | ND | ND | ND | ND | ND | ND | 0.13 |
| Degradation Product | | | | | | | |
| a) Deamidation | | | | | | | |
| Any Unidentified | ND | ND | ND | ND | ND | ND | ND |
| Unspecified | | | | | | | |
| Degradation Product | | | | | | | |
| Total Related Substances | ND | ND | ND | ND | ND | ND | 0.13 |
| pH | 3.28 | 3.27 | NT | 3.40 | 3.38 | NT | NT |

ND = Not Detected
NT = Not Tested

TABLE 14
Stability of Cetrorelix Injection with 0.25% w/v Acetic Acid Filled in Syringes and Stored at 25° C. for 9 Months

| | Time Point | | | | | |
|---|---|---|---|---|---|---|
| | T = 0 | 1 Month | 2 Months | 3 Months | 6 Months | 9 Months |
| Test | 21 Apr. 2022 | 14 May 2022 | 14 Jun. 2022 | 14 Jul. 2022 | 14 Oct. 2022 | 14 Jan. 2023 |
| Physical Appearance | Clear, colorless | Clear, colorless | Clear, colorless | Clear, colorless | Clear, colorless | Clear, colorless |
| | solution free of | solution free of | solution free of | solution free of | solution free of | solution free of |
| | visible | visible | visible | visible | visible | visible |
| | particulates | particulates | particulates | particulates | particulates | particulates |
| Assay, % Label Claim | 100.0 | 102.6 | 102.1 | 101.3 | 95.5 | 94.7 |
| (0.25 mg/mL) | | | | | | |

Related Substances via HPLC (w/w %)

| | | | | | | |
|---|---|---|---|---|---|---|
| Specified-Identified | <LOQ | 0.22 | 0.43 | 0.58 | 1.21 | 1.36 |
| Degradation Product | | | | | | |
| a) Deamidation | | | | | | |
| Any Unidentified | ND | RRT 0.86-0.07 | RRT 0.86-0.15 | RRT 0.86-0.22 | RRT 0.86-0.44 | RRT 0.86-0.50 |
| Unspecified | | | | | | RRT 1.27-0.14 |
| Degradation Product | | | | | | |
| Total Related Substances | ND | 0.29 | 1.08 | 0.86 | 1.76 | 2.12 |
| pH | 3.19 | 3.39 | NT | 3.17 | 3.14 | 3.18 |

ND = Not Detected
NT = Not Tested

TABLE 15
Stability of Cetrorelix Injection with 0.5% w/v and 0.8% w/v Acetic Acid Stored at 25° C. for 3 Months

| | Time Point | | | | | |
|---|---|---|---|---|---|---|
| | 1 Month | | 2 Months | | 3 Months | |
| | 0.5% AA | 0.8% AA | 0.5% AA | 0.8% AA | 0.5% AA | 0.8% AA |
| Test | 14 May 2022 | | 14 Jun. 2022 | | 14 Jul. 2022 | |
| Physical Appearance | Clear, colorless | Clear, colorless | Clear, colorless | Clear, colorless | Clear, colorless | Clear, colorless |
| | solution free of | solution free of | solution free of | solution free of | solution free of | solution free of |
| | visible | visible | visible | visible | visible | visible |
| | particulates | particulates | particulates | particulates | particulates | particulates |
| Assay, % Label Claim | 103.0 | 104.6 | 102.3 | 103.9 | 101.0 | 102.6 |
| (0.25 mg/mL) | | | | | | |

Related Substances via HPLC (w/w %)

| | | | | | | |
|---|---|---|---|---|---|---|
| Specified-Identified | 0.29 | 0.42 | 0.62 | 0.84 | 0.88 | 1.29 |
| Degradation Product | | | | | | |
| a) Deamidation | | | | | | |
| Any Unidentified | RRT 0.86-0.07 | RRT 0.86-0.08 | RRT 0.52-0.06 | RRT 0.52-0.08 | RRT 0.52-0.12 | RRT 0.52-0.12 |
| Unspecified | | | RRT 0.86-0.16 | RRT 0.86-0.17 | RRT 0.86-0.22 | RRT 0.86-0.22 |
| Degradation Product | | | | | | |
| Total Related Substances | 0.36 | 0.50 | 0.85 | 1.09 | 1.20 | 1.63 |
| pH | 3.03 | 2.96 | NT | NT | 2.95 | 2.76 |

ND = Not Detected
NT = Not Tested

TABLE 16
Stability of Cetrorelix Injection with 0.25% w/v Acetic Acid Filled in Syringes and Stored at 2-8° C. for 11 Months

| | Time Point | | | | | |
|---|---|---|---|---|---|---|
| | T = 0 | 1 Month | 3 Months | 6 Months | 9 Months | 11 Months |
| Test | 21 Apr. 2022 | 14 May 2022 | 14 Jul. 2022 | 14 Oct. 2022 | 14 Jan. 2023 | 14 Mar. 2023 |
| Physical Appearance | Clear, colorless | Clear, colorless | Clear, colorless | Clear, colorless | Clear, colorless | Clear, colorless |
| | solution free of | solution free of | solution free of | solution free of | solution free of | solution free of |
| | visible | visible | visible | visible | visible | visible |
| | particulates | particulates | particulates | particulates | particulates | particulates |
| Assay, % Label Claim | 100.0 | 102.3 | 101.8 | 96.2 | 96.4 | 96.7 |
| (0.25 mg/mL) | | | | | | |

Related Substances via HPLC (w/w %)

| | | | | | | |
|---|---|---|---|---|---|---|
| Specified-Identified | ND | ND | ND | ND | 0.15 | 0.16 |
| Degradation Product | | | | | | |
| a) Deamidation | | | | | | |
| Any Unidentified | ND | ND | ND | ND | RRT 0.86-<LOQ | ND |
| Unspecified | | | | | | |
| Degradation Product | | | | | | |
| Total Related Substances | ND | ND | ND | ND | 0.15 | 0.16 |
| pH | 3.19 | 3.28 | 3.15 | 3.13 | 3.14 | NT |

ND = Not Detected
NT = Not Tested

TABLE 17
Stability of Cetrorelix Injection with 0.5% w/v and 0.8% w/v Acetic Acid Stored at 2-8° C. for 6 Months

| | Time Point | | | | | |
|---|---|---|---|---|---|---|
| | 1 Month | | 3 Months | | 6 Months | |
| | 0.5% AA | 0.8% AA | 0.5% AA | 0.8% AA | 0.5% AA | 0.8% AA |
| Test | 14 May 2022 | | 14 Jul. 2022 | | 14 Oct. 2022 | |
| Physical Appearance | Clear, colorless | Clear, colorless | Clear, colorless | Clear, colorless | Clear, colorless | Clear, colorless |
| | solution free of | solution free of | solution free of | solution free of | solution free of | solution free of |
| | visible | visible | visible | visible | visible | visible |
| | particulates | particulates | particulates | particulates | particulates | particulates |
| Assay, % Label Claim | 102.6 | 105.6 | 102.3 | 103.4 | 96.7 | 98.7 |
| (0.25 mg/mL) | | | | | | |

Related Substances via HPLC (w/w %)

| | | | | | | |
|---|---|---|---|---|---|---|
| Specified-Identified | ND | ND | ND | ND | ND | 0.21 |
| Degradation Product | | | | | | |
| a) Deamidation | | | | | | |
| Any Unidentified | ND | ND | ND | ND | ND | ND |
| Unspecified | | | | | | |
| Degradation Product | | | | | | |
| Total Related Substances | ND | ND | ND | ND | ND | 0.21 |
| pH | 3.03 | 2.95 | 2.88 | 2.77 | 2.89 | 2.78 |

ND = Not Detected
NT = Not Tested

TABLE 18
Stability of Cetrorelix Injection with 0.25% w/v Acetic pH Adjusted to pH 4.0 Stored at 25° C. for 9 Months

| | Time Point | | | | | |
|---|---|---|---|---|---|---|
| | T = 0 | 1 Month | 2 Months | 3 Months | 6 Months | 9 Months |
| Test | 21 Apr. 2022 | 14 May 2022 | 14 Jun. 2022 | 14 Jul. 2022 | 14 Oct. 2022 | 14 Jan. 2023 |
| Physical Appearance | Clear, colorless | Clear, colorless | Clear, colorless | Clear, colorless | Clear, colorless | Clear, colorless |
| | solution free of | solution free of | solution free of | solution free of | solution free of | solution free of |
| | visible | visible | visible | visible | visible | visible |
| | particulates | particulates | particulates | particulates | particulates | particulates |
| Assay, % Label Claim | 96.9 | 101.5 | 102.0 | 94.0 | 95.7 | 93.5 |
| (0.25 mg/mL) | | | | | | |

Related Substances via HPLC (w/w %)

| | | | | | | |
|---|---|---|---|---|---|---|
| Specified-Identified | <LOQ | ND | 0.06 | 0.13 | 0.24 | 0.21 |
| Degradation Product | | | | | | |
| a) Deamidation | | | | | | |
| Any Unidentified | ND | RRT 0.86-0.07 | RRT 0.86-0.13 | RRT 0.86-0.18 | RRT 0.86-0.35 | RRT 0.86-0.40 |
| Unspecified | | | | | | RRT 1.27-0.19 |
| Degradation Product | | | | | | |
| Total Related Substances | ND | 0.07 | 0.20 | 0.31 | 0.59 | 0.80 |
| pH | 4.08 | 4.34 | NT | 4.00 | 4.02 | 4.01 |

ND = Not Detected
NT = Not Tested

TABLE 19
Stability of Cetrorelix Injection with 0.25% w/v Acetic pH Adjusted to pH 4.0 Stored at 2-8° C. for 11 Months

| | Time Point | | | | | |
|---|---|---|---|---|---|---|
| | T = 0 | 1 Month | 3 Months | 6 Months | 9 Months | 11 Months |
| Test | 21 Apr. 2022 | 14 May 2022 | 14 Jul. 2022 | 14 Oct. 2022 | 14 Jan. 2023 | 14 Mar. 2023 |
| Physical Appearance | Clear, colorless | Clear, colorless | Clear, colorless | Clear, colorless | Clear, colorless | Clear, colorless |
| | solution free of | solution free of | solution free of | solution free of | solution free of | solution free of |
| | visible | visible | visible | visible | visible | visible |
| | particulates | particulates | particulates | particulates | particulates | particulates |
| Assay, % Label Claim | 96.9 | 97.6 | 97.3 | 96.0 | 94.1 | 93.2 |
| (0.25 mg/mL) | | | | | | |

Related Substances via HPLC (w/w %)

| | | | | | | |
|---|---|---|---|---|---|---|
| Specified-Identified | <LOQ | ND | ND | ND | <LOQ | ND |
| Degradation Product | | | | | | |
| a) Deamidation | | | | | | |
| Any Unidentified | ND | ND | ND | ND | ND | ND |
| Unspecified | | | | | | |
| Degradation Product | | | | | | |
| Total Related Substances | ND | ND | ND | ND | ND | ND |
| pH | 4.08 | 4.33 | 4.02 | 4.02 | 4.01 | NT |

ND = Not Detected
NT = Not Tested

TABLE 20
Stability of Cetrorelix Injection pH 4.5, pH 5.0, pH 6.0 Stored at 25° C. for 5 Months

| | Time Point | | | | | | | | |
|---|---|---|---|---|---|---|---|---|---|
| | 1 Month | | | 3 Months | | | 5 Months | | |
| | pH 4.5 | pH 5.0 | pH 6.0 | pH 4.5 | pH 5.0 | pH 6.0 | pH 4.5 | pH 5.0 | pH 6.0 |
| Test | 8 Sep. 2022 | | | 8 Nov. 2022 | | | 8 Jan. 2023 | | |
| Physical Appearance | Clear, | Clear, | Clear, | Clear, | Clear, | Clear, | Clear, | Clear, | Clear, |
| | colorless | colorless | colorless | colorless | colorless | colorless | colorless | colorless | colorless |
| | solution | solution | solution | solution | solution | solution | solution | solution | solution |
| | free of | free of | free of | free of | free of | free of | free of | free of | free of |
| | visible | visible | visible | visible | visible | visible | visible | visible | visible |
| | partic- | partic- | partic- | partic- | partic- | partic- | partic- | partic- | partic- |
| | ulates | ulates | ulates | ulates | ulates | ulates | ulates | ulates | ulates |
| Assay, % Label Claim | 93.4 | 100.4 | 93.7 | 99.3 | 98.9 | 96.7 | 95.7 | 95.8 | 94.2 |
| (0.25 mg/mL) | | | | | | | | | |

Related Substances via HPLC (w/w %)

| | | | | | | | | | |
|---|---|---|---|---|---|---|---|---|---|
| Specified-Identified | ND | ND | ND | ND | ND | ND | 0.06 | 0.06 | 0.20 |
| Degradation Product | | | | | | | | | |
| a) Deamidation | | | | | | | | | |
| Any Unidentified | ND | ND | ND | RRT 0.86- | RRT 0.86- | ND | RRT 0.86- | RRT 0.86- | RRT 0.86- |
| Unspecified | | | | 0.16 | 0.11 | | 0.23 | 0.15 | 0.12 |
| Degradation Product | | | | | | | RRT 1.27- | | |
| | | | | | | | 0.06 | | |
| Total Related Substances | ND | ND | ND | 0.16 | 0.11 | ND | 0.35 | 0.21 | 0.32 |
| pH | NT | NT | NT | 4.53 | 4.92 | 5.96 | 4.48 | 4.97 | 5.92 |

ND = Not Detected
NT = Not Tested

TABLE 21
Stability of Cetrorelix Injection with 0.1% w/v Acetic pH Adjusted to pH 4.5 Stored at 2-8 C. ° for 7.5 Months

| | Time Point | | | | | |
|---|---|---|---|---|---|---|
| | | | | | 7.5 Months | |
| | T = 0 | 1 Month | 3 Months | 5 Months | Sample 1 | Sample 2 |
| Test | 8 Aug. 2022 | 8 Sep. 2022 | 8 Nov. 2022 | 8 Jan. 2023 | 22 Mar. 2023 | |
| Physical Appearance | Clear, colorless | Clear, colorless | Clear, colorless | Clear, colorless | Clear, colorless | Clear, colorless |
| | solution free of | solution free of | solution free of | solution free of | solution free of | solution free of |
| | visible | visible | visible | visible | visible | visible |
| | particulates | particulates | particulates | particulates | particulates | particulates |
| Assay, % Label Claim | 98.8 | 93.0 | 98.5 | 95.8 | 94.8 | 94.7 |
| (0.25 mg/mL) | | | | | | |

Related Substances via HPLC (w/w %)

| | | | | | | |
|---|---|---|---|---|---|---|
| Specified-Identified | ND | ND | ND | <LOQ | ND | ND |
| Degradation Product | | | | | | |
| a) Deamidation | | | | | | |
| Any Unidentified | ND | ND | ND | RRT 0.86-<LOQ | ND | ND |
| Unspecified | | | | | | |
| Degradation Product | | | | | | |
| Total Related Substances | ND | ND | ND | ND | ND | ND |
| pH | 4.48 | NT | NT | 4.49 | NT | NT |

ND = Not Detected
NT = Not Tested

TABLE 22
Stability of Cetrorelix Injection with 0.1% w/v Acetic pH Adjusted to pH 5.0 Stored at 2-8 C. ° C. for 7.5 Months

| | Time Point | | | | | |
|---|---|---|---|---|---|---|
| | | | | | 7.5 Months | |
| | T = 0 | 1 Month | 3 Months | 5 Months | Sample 1 | Sample 2 |
| Test | 8 Aug. 2022 | 8 Sep. 2022 | 8 Nov. 2022 | 8 Jan. 2023 | 22 Mar. 2023 | |
| Physical Appearance | Clear, colorless | Clear, colorless | Clear, colorless | Clear, colorless | Clear, colorless | Clear, colorless |
| | solution free of | solution free of | solution free of | solution free of | solution free of | solution free of |
| | visible | visible | visible | visible | visible | visible |
| | particulates | particulates | particulates | particulates | particulates | particulates |
| Assay, % Label Claim | 101.1 | 92.5 | 97.5 | 96.4 | 95.3 | 95.9 |
| (0.25 mg/mL) | | | | | | |

Related Substances via HPLC (w/w %)

| | | | | | | |
|---|---|---|---|---|---|---|
| Specified-Identified | ND | ND | ND | <LOQ | ND | ND |
| Degradation Product | | | | | | |
| a) Deamidation | | | | | | |
| Any Unidentified | ND | ND | ND | RRT 0.86-<LOQ | ND | ND |
| Unspecified | | | | | | |
| Degradation Product | | | | | | |
| Total Related Substances | ND | ND | ND | ND | ND | ND |
| pH | 4.98 | NT | NT | 4.97 | NT | NT |

ND = Not Detected
NT = Not Tested

TABLE 23
Stability of Cetrorelix Injection with 0.1% w/v Acetic pH Adjusted to pH 6.0 Stored at 2-8 C. ° C. for 7.5 Months

| | Time Point | | | | | |
|---|---|---|---|---|---|---|
| | | | | | 7.5 Months | |
| | T = 0 | 1 Month | 3 Months | 5 Months | Sample 1 | Sample 2 |
| Test | 8 Aug. 2022 | 8 Sep. 2022 | 8 Nov. 2022 | 8 Jan. 2023 | 22 Mar. 2023 | |
| Physical Appearance | Clear, colorless | Clear, colorless | Clear, colorless | Clear, colorless | Clear, colorless | Clear, colorless |
| | solution free of | solution free of | solution free of | solution free of | solution free of | solution free of |
| | visible | visible | visible | visible | visible | visible |
| | particulates | particulates | particulates | particulates | particulates | particulates |
| Assay, % Label Claim | 99.9 | 96.3 | 100.4 | 94.4 | 93.8 | 93.9 |
| (0.25 mg/mL) | | | | | | |

Related Substances via HPLC (w/w %)

| | | | | | | |
|---|---|---|---|---|---|---|
| Specified-Identified | ND | ND | ND | <LOQ | ND | ND |
| Degradation Product | | | | | | |
| a) Deamidation | | | | | | |
| Any Unidentified | ND | ND | ND | RRT 0.86-<LOQ | ND | ND |
| Unspecified | | | | | | |
| Degradation Product | | | | | | |
| Total Related Substances | ND | ND | ND | ND | ND | ND |
| pH | 6.01 | NT | NT | 5.93 | NT | NT |

ND = Not Detected
NT = Not Tested

In view of the above, it will be seen that several objectives of the invention are achieved, and other advantages attained.

As various changes could be made in the above methods and compositions without departing from the scope of the invention, it is intended that all matter contained in the above description and shown in the accompanying drawings shall be interpreted as illustrative and not in a limiting sense.

All references cited in this specification, including but not limited to patent publications and non-patent literature, and references cited therein, are hereby incorporated by reference. The discussion of the references herein is intended merely to summarize the assertions made by the authors and no admission is made that any reference constitutes prior art. Applicants reserve the right to challenge the accuracy and pertinence of the cited references.

As used herein, in particular embodiments, the terms “about” or “approximately” when preceding a numerical value indicates the value plus or minus a range of 10%. Where a range of values is provided, it is understood that each intervening value, to the tenth of the unit of the lower limit unless the context clearly dictates otherwise, between the upper and lower limit of that range and any other stated or intervening value in that stated range is encompassed within the disclosure. That the upper and lower limits of these smaller ranges can independently be included in the smaller ranges is also encompassed within the disclosure, subject to any specifically excluded limit in the stated range. Where the stated range includes one or both of the limits, ranges excluding either or both of those included limits are also included in the disclosure.

The indefinite articles “a” and “an,” as used herein in the specification and in the embodiments, unless clearly indicated to the contrary, should be understood to mean “at least one.”

The phrase “and/or,” as used herein in the specification and in the embodiments, should be understood to mean “either or both” of the elements so conjoined, i.e., elements that are conjunctively present in some cases and disjunctively present in other cases. Multiple elements listed with “and/or” should be construed in the same fashion, i.e., “one or more” of the elements so conjoined. Other elements can optionally be present other than the elements specifically identified by the “and/or” clause, whether related or unrelated to those elements specifically identified. Thus, as a non-limiting example, a reference to “A and/or B”, when used in conjunction with open-ended language such as “comprising” can refer, in one embodiment, to A only (optionally including elements other than B); in another embodiment, to B only (optionally including elements other than A); in yet another embodiment, to both A and B (optionally including other elements); etc.

As used herein in the specification and in the embodiments, “or” should be understood to have the same meaning as “and/or” as defined above. For example, when separating items in a list, “or” or “and/or” shall be interpreted as being inclusive, i.e., the inclusion of at least one, but also including more than one, of a number or list of elements, and, optionally, additional unlisted items. Only terms clearly indicated to the contrary, such as “only one of” or “exactly one of,” or, when used in the embodiments, “consisting of,” will refer to the inclusion of exactly one element of a number or list of elements. In general, the term “or” as used herein shall only be interpreted as indicating exclusive alternatives (i.e. “one or the other but not both”) when preceded by terms of exclusivity, such as “either,” “one of,” “only one of,” or “exactly one of.” “Consisting essentially of,” when used in the embodiments, shall have its ordinary meaning as used in the field of patent law.

As used herein in the specification and in the embodiments, the phrase “at least one,” in reference to a list of one or more elements, should be understood to mean at least one element selected from any one or more of the elements in the list of elements, but not necessarily including at least one of each and every element specifically listed within the list of elements and not excluding any combinations of elements in the list of elements. This definition also allows that elements can optionally be present other than the elements specifically identified within the list of elements to which the phrase “at least one” refers, whether related or unrelated to those elements specifically identified. Thus, as a non-limiting example, “at least one of A and B” (or, equivalently, “at least one of A or B,” or, equivalently “at least one of A and/or B”) can refer, in one embodiment, to at least one, optionally including more than one, A, with no B present (and optionally including elements other than B); in another embodiment, to at least one, optionally including more than one, B, with no A present (and optionally including elements other than A); in yet another embodiment, to at least one, optionally including more than one, A, and at least one, optionally including more than one, B (and optionally including other elements); etc.

What is claimed is:

1. An aqueous formulation comprising cetrorelix and/or a salt thereof, an isotonicity adjuster, a pH adjuster and water, suitable for subcutaneous injection, wherein the aqueous formulation further comprises less than 0.5% deamidated cetrorelix (Ac-D-2-Nal-D-Cpa-D-Pal-Ser-Tyr-D-Cit-Leu-Arg-Pro-D-Ala-OH) after storage for at least one month at 25° C.

2. The aqueous formulation of claim 1, comprising cetrorelix acetate.

3. The aqueous formulation of claim 1, further comprising less than 0.5% of deamidated cetrorelix after storage for at least two months at 25° C.

4. The aqueous formulation of claim 1, further comprising less than 0.5% of deamidated cetrorelix after storage for at least four months at 25° C.

5. The aqueous formulation of claim 1, further comprising less than 0.5% of any non-cetrorelix impurity after storage for at least three months at 25° C.

6. The aqueous formulation of claim 1, further comprising less than 0.5% of deamidated cetrorelix after storage for at least three months at 2-8° C.

7. The aqueous formulation of claim 1, further comprising less than 0.5% of deamidated cetrorelix after storage for at least six months at 2-8° C.

8. The aqueous formulation of claim 1, further comprising less than 0.5% of deamidated cetrorelix after storage for at least twelve months at 2-8° C.

9. The aqueous formulation of claim 1, further comprising less than 0.5% of deamidated cetrorelix after storage for at least fifteen months at 2-8° C.

10. The aqueous formulation of claim 1, further comprising less than 0.1% of any non-cetrorelix impurity after storage for at least twelve months at 2-8° C.

11. The aqueous formulation of claim 1, further comprising less than or equal to 0.1% of any non-cetrorelix impurity after storage for at least fifteen months at 2-8° C.

12. The aqueous formulation of claim 1, wherein the isotonicity adjuster is mannitol.

13. The aqueous formulation of claim 1, wherein the pH adjuster is acetic acid.

14. The aqueous formulation of claim 1, wherein the pH adjuster is NaOH.

15. The aqueous formulation of claim 1, having a pH of 2.5-6.0.

16. The aqueous formulation of claim 1, having a pH of 4.0-6.0.

17. The aqueous formulation of claim 1, packaged in a syringe.

18. The aqueous formulation of claim 1, packaged in a vial.

19. The aqueous formulation of claim 1, having a cetrorelix concentration of 0.20-0.30 mg/mL.

20. A method of treating a patient with cetrorelix comprising injecting the aqueous formulation of a claim 1 to the patient in a manner sufficient to treat the patient.
